(19) United States
(12) Patent Application Publication
(10) Pub. No.: US 2026/0083562 A1
(43) Pub. Date: Mar. 26, 2026

(54) ADJUSTABLE INTERATRIAL DEVICES

(71) Applicant: Shifamed Holdings, LLC, Campbell, CA (US)

(72) Inventors: Brian Fahey, Mountain View, CA (US); William Jason Fox, San Mateo, CA (US)

(21) Appl. No.: 19/403,121

(22) Filed: Nov. 27, 2025

Related U.S. Application Data
(63) Continuation of application No. 17/783,579.
(60) Provisional application No. 62/952,075, filed on Dec. 20, 2019.
(60) Provisional application No. 62/952,109, filed on Dec. 20, 2019.

(51) Int. Cl. A61F 2/24; A61B 5/00; A61B 5/0215

(52) U.S. Cl. CPC A61F 2/2476; A61B 5/02158; A61B 5/686; A61B 5/6869; A61B 2562/0247; A61F 2210/0033; A61F 2220/0008; A61F 2250/0013

(57) ABSTRACT

Closure devices that selectively control blood flow between the right atrium and the left atrium of a heart of a patient. The control of blood flow can be based on detected values from a pressure sensor. The closure devices can include a body configured to engage native tissue when the device is deployed across the septal wall. In a first state at least a portion of (an inner surface of) a lumen is closed, preventing blood flow through the closure device. In a second state, the lumen is open to enable blood flow from the left atrium to the right atrium. A closure element can be selectively adjusted to control the flow of blood through the lumen. A volume of an expandable chamber can be increased or decreased to place the closure device into the first state or the second state.

### CROSS-REFERENCE TO RELATED APPLICATION(S)

This is a continuation of U.S. patent application Ser. No. 17/783,579, filed Jun. 8, 2022, which is a 35 U.S.C. § 371 U.S. National Phase application of International Patent Application No. PCT/US2020/064529, filed Dec. 11, 2020, which claims the benefit of the following pending applications:
(a) U.S. Provisional Patent App. No. 62/952,109, filed Dec. 20, 2019; and
(b) U.S. Provisional Patent App. No. 62/952,075, filed Dec. 20, 2019.

The foregoing applications are incorporated herein by reference in their entireties. Further, components and features of embodiments disclosed in the applications incorporated by reference may be combined with various components and features disclosed and claimed in the present application.

### TECHNICAL FIELD

The present technology generally relates to implantable medical devices, and in particular to implantable interatrial devices that selectively control blood flow between the right atrium and the left atrium of a heart.

### BACKGROUND

Heart failure is a medical condition associated with the inability of the heart to effectively pump blood to the body. Heart failure affects millions of people worldwide, and may arise from multiple root causes, but is generally associated with myocardial stiffening, myocardial shape remodeling, and/or abnormal cardiovascular dynamics. Chronic heart failure is a progressive disease that worsens considerably over time. Initially, the body's autonomic nervous system adapts to heart failure by altering the sympathetic and parasympathetic balance. While these adaptations are helpful in the short-term, over a longer period of time they may serve to make the disease worse.

Heart failure (HF) is a medical term that includes both heart failure with reduced ejection fraction (HFrEF) and heart failure with preserved ejection fraction (HFpEF). The prognosis with both HFpEF and HFrEF is poor; one-year mortality is 26% and 22%, respectively, according to one epidemiology study. In spite of the high prevalence of HFpEF, there remain limited options for HFpEF patients. Pharmacological therapies have been shown to impact mortality in HFrEF patients, but there are no similarly-effective evidence-based pharmacotherapies for treating HFpEF patients. Current practice is to manage and support patients while their health continues to decline.

A common symptom among heart failure patients is elevated left atrial pressure. In the past, clinicians have treated patients with elevated left atrial pressure by creating a shunt between the left and right atria using a blade or balloon septostomy. The shunt decompresses the left atrium (LA) by relieving pressure to the right atrium (RA) and systemic veins. Over time, however, the shunt typically will close or reduce in size. More recently, percutaneous interatrial shunt devices have been developed which have been shown to effectively reduce left atrial pressure. However, these percutaneous devices often have an annular passage with a fixed diameter which fails to account for a patient's changing physiology and condition. For this reason, existing percutaneous shunt devices may have a diminishing clinical effect after a period of time. Many existing percutaneous shunt devices typically are also only available in a single size that may work well for one patient but not another. Also, sometimes the amount of shunting created during the initial procedure is later determined to be less than optimal months later. Accordingly, there is a need for improved devices, systems, and methods for treating heart failure patients, particularly those with elevated left atrial pressure.

### BRIEF DESCRIPTION OF THE DRAWINGS

FIG. 1 is a schematic illustration of an interatrial device implanted in a heart and configured in accordance with select embodiments of the present technology;

FIG. 2 illustrates an adjustable closure device configured in accordance with select embodiments of the present technology;

FIGS. 3A and 3B are cross-sectional illustrations of the adjustable closure device shown in FIG. 2 and configured in accordance with select embodiments of the present technology;

FIG. 4 is a schematic illustration of an adjustable closure device having sensing in accordance with select embodiments of the present technology

FIG. 5 is a schematic illustration of an operation of an adjustable closure device in accordance with select embodiments of the present technology;

FIGS. 6A-6C are cross-sectional schematic illustrations of an adjustable closure device configured in accordance with select embodiments of the present technology;

FIGS. 7A and 7B are cross-sectional schematic illustrations of an adjustable closure device configured in accordance with select embodiments of the present technology; and

FIGS. 8A and 8B are cross-sectional schematic illustrations of an adjustable closure device configured in accordance with select embodiments of the present technology.

### DETAILED DESCRIPTION

The present technology is directed to implantable closure devices (“adjustable closure device,” “closure device”) that selectively prevent or permit blood flow between the left atrium and the right atrium of a patient. In embodiments disclosed herein, the adjustable interatrial closure devices include a closure element configured to control (e.g., prevent or promote) fluid flow through the device, and an actuator operably coupled to the closure element and configured to move or translate the closure element between a plurality of positions or arrangements to selectively control blood flow between the left atrium and the right atrium. In some embodiments, a closure device comprises a tubular element having an outer surface configured to engage native tissue, and an inner surface spanning the tubular element from the left atrium to the right atrium. In a first state the inner surface is closed, substantially preventing fluid flow through the closure device. Reference throughout this specification to “preventing fluid flow” or “substantially preventing fluid flow” mean that (a) only negligible flow is allowed, (b) the amount of flow is not clinically relevant, and/or (c) the shunt is in a fully closed position. In a second state, the inner surface is open (e.g., defining a lumen), enabling blood to flow from the left atrium to the right atrium when the device is deployed across the septal wall.

The perimeter of the inner surface can be selectively adjusted by the closure element to control (e.g., prevent or permit) the flow of blood therethrough. In some embodiments, the outer surface of the device is configured to retain a substantially constant outer perimeter as the inner surface (e.g., perimeter) is selectively adjusted. In some embodiments, the closure device comprises one or more sensors configured to detect one or more physiological properties (e.g., parameters) of the patient—e.g., left atrial pressure, right atrial pressure, and/or heart rate. In some embodiments, a closure device is implanted in the first state and one or more sensors are detecting physiological parameter(s) of the patient. The closure device may be adjusted (e.g., converted) to the second state considering (e.g., based on) the detected physiological parameter(s) of the patient.

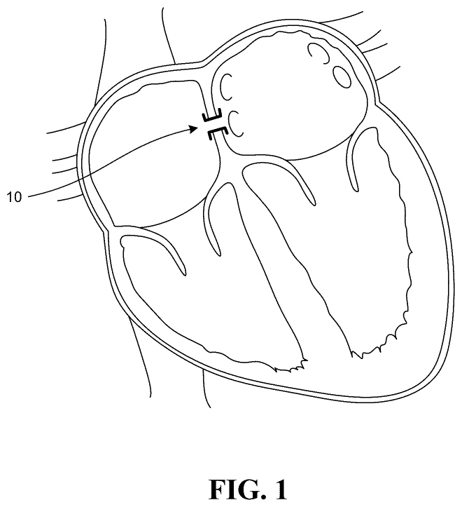

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1-8B.

In some embodiments, a closure device is implanted to close an opening that is formed during an interventional cardiac procedure. The interventional cardiac procedure can be a procedure that is performed in or adjacent to the left atrium and/or the left ventricle. The interventional procedure may be performed in a minimally invasive manner (e.g., via catheterization). Examples of interventional procedures that include transseptal puncture, and for which the closure device may be implanted, include: (i) left atrial appendage ligation, (ii) mitral valve repair, (iii) mitral valve replacement, and (iv) ablation of a variety of arrhythmias—for example, ablation for LA or left ventricular tachycardias, pulmonary vein isolation (PVI) in patients with atrial fibrillation (AF), or ablation of the atrioventricular node. In some embodiments, the closure device is implanted using a separate (e.g., distinct) delivery system (e.g., catheter). In some embodiments, the closure device is implanted without removal of a (e.g., any) catheter inserted for performing the interventional cardiac procedure. The closure device may be implanted in a first state, for example a closed state for preventing fluid flow through the (e.g., transseptal) opening formed during the interventional cardiac procedure. In some embodiments, the closure device is adjustable to a second state at a time following implantation. The adjustment may be made by (e.g., balloon) dilation of the closure device. The dilation may establish a lumen in the closure device (e.g., to form a shunt). The lumen may be established at a pre-determined geometry (e.g., having a cross-section that is ovular, circular, rectangular, or star shaped) and/or size (e.g., 5 mm, 6 mm, 7 mm, 8 mm, 9 mm, or 10 mm). Once established, the closure device may maintain the lumen at the pre-determined geometry and/or size (e.g., indefinitely). Following implantation, a host response may include tissue overgrowth (e.g., pannus and/or endothelialization) of the closure device at the implanted location. In some embodiments, an adjustment of the closure device from the first state to the second state may include a filtration device to block and/or capture any dislodged tissue and/or thrombi from entering the circulatory system of the patient.

Reference throughout this specification to “one embodiment” or “an embodiment” means that a particular feature, structure or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “substantially,” “approximately,” and “about” are used herein to mean the stated value plus or minus 10%.

As used herein, in various embodiments, the terms “interatrial device,” “interatrial shunt device,” “IAD,” “IASD,” “interatrial shunt,” and “shunt” are used interchangeably and, in at least one configuration, refer to a shunting element that provides a blood flow between a first region (e.g., a LA of a heart) and a second region (e.g., a RA or coronary sinus of the heart) of a patient. Although described in terms of a shunt between the atria, namely the LA and the RA, one will appreciate that the technology may be applied equally to other medical devices. For example, the shunt may be positioned between other chambers and passages of the heart or other parts of the cardiovascular system. For example, any of the shunts described herein, including those referred to as “interatrial,” may be nevertheless used and/or modified to shunt between the LA and the coronary sinus, or between the right pulmonary vein and the superior vena cava. Moreover, while the disclosure herein primarily describes shunting blood from the LA to the RA, the present technology can be readily adapted to shunt blood from the RA to the LA to treat certain conditions, such as pulmonary hypertension. For example, mirror images of embodiments, or in some cases identical embodiments, used to shunt blood from the LA to the RA can be used to shunt blood from the RA to the LA in certain patients. In another example, the shunt may be used to facilitate flow between an organ and organ, organ and vessel, etc. The shunt may also be used for fluids other than blood.

The technologies described herein may be used for an ophthalmology shunt to flow aqueous or fluids to treat gastrointestinal disorders. The technologies described herein may also be used for controlled delivery of other fluids such as saline, drugs, or pharmacological agents.

The headings provided herein are for convenience only and do not interpret the scope or meaning of the claimed present technology.

### Interatrial Shunt for Treatment of Heart Failure

Heart failure can be classified into one of at least two categories based upon the ejection fraction a patient experiences: (1) HFpEF, historically referred to as diastolic heart failure or (2) HFrEF, historically referred to as systolic heart failure. One definition of HFrEF is a left ventricular ejection fraction lower than 35%-40%. Though related, the underlying pathophysiology and the treatment regimens for each heart failure classification may vary considerably. For example, while there are established pharmaceutical therapies that can help treat the symptoms of HFrEF, and at times slow or reverse the progression of the disease, there are limited available pharmaceutical therapies for HFpEF with only questionable efficacy.

In heart failure patients, abnormal function in the left ventricle (LV) leads to pressure build-up in the LA. This leads directly to higher pressures in the pulmonary venous system, which feeds the LA. Elevated pulmonary venous pressures push fluid out of capillaries and into the lungs. This fluid build-up leads to pulmonary congestion and many of the symptoms of heart failure, including shortness of breath and signs of exertion with even mild physical activity. Risk factors for HF include renal dysfunction, hypertension, hyperlipidemia, diabetes, smoking, obesity, old age, and obstructive sleep apnea. HF patients can have increased stiffness of the LV which causes a decrease in left ventricular relaxation during diastole resulting in increased pressure and inadequate filling of the ventricle. HF patients may also have an increased risk for atrial fibrillation and pulmonary hypertension, and typically have other comorbidities that can complicate treatment options.

Interatrial shunts have recently been proposed as a way to reduce elevated left atrial pressure, and this emerging class of cardiovascular therapeutic interventions has been demonstrated to have significant clinical promise. FIG. 1, for example, shows the conventional placement of a shunt in the septal wall between the LA and RA. Most conventional interatrial shunts (e.g., shunt 10) involve creating a hole or inserting an implant with a lumen into the atrial septal wall, thereby creating a fluid communication pathway between the LA and the RA. As such, elevated left atrial pressure may be partially relieved by unloading the LA into the RA. In early clinical trials, this approach has been shown to improve symptoms of heart failure.

One challenge with many conventional interatrial shunts is determining the most appropriate size and shape of the shunt lumen. A lumen that is too small may not adequately unload the LA and relieve symptoms; a lumen that is too large may overload the RA and right-heart more generally, creating new problems for the patient. Moreover, the relationship between pressure reduction and clinical outcomes and the degree of pressure reduction required for optimized outcomes is still not fully understood, in part because the pathophysiology for HFpEF (and to a lesser extent, HFrEF) is not completely understood. As such, clinicians are forced to take a best guess at selecting the appropriately sized shunt (based on limited clinical evidence) and generally cannot adjust the sizing over time. Worse, clinicians must select the size of the shunt based on general factors (e.g., the size of the patient's anatomical structures, the patient's hemodynamic measurements taken at one snapshot in time, etc.) and/or the design of available devices rather than the individual patient's health and anticipated response. With traditional devices, the clinician does not have the ability to adjust or titrate the therapy once the device is implanted, for example, in response to changing patient conditions such as progression of disease. By contrast, interatrial shunting systems configured in accordance with embodiments of the present technology allow a clinician to select the size-perioperatively or post-implant-based on the patient.

A further challenge with conventional interatrial shunts is that function of the LA (and more generally, the cardiovascular system) can vary depending on a number of factors, for example during exercise, during periods where a patient's medication adherence has slipped, as the patient's disease progresses, or during other periods. Existing conventional shunts are generally static in nature and lack an ability to adapt to patient conditions in such a way to optimize therapy.

Other shortcomings of existing conventional interatrial shunts include: (1) shunts tending to be permanently implanted in the septal wall in a way that complicates or prevents future transseptal access, which may prohibit or complicate additional left-heart procedures that generally would require transseptal access; (2) shunts tending to be fixed and unable to adapt to changing patient conditions, such as progression of disease, and (3) a lack of sensors and/or machine-learning capability that limit the information available from the patient and limit the ability to improve therapy for the patient (or for the larger patient cohort) over time.

### Adjustable Interatrial Closure Devices

As provided above, the present technology is generally directed to interatrial closure devices implantable into a patient at or adjacent a septal wall. The interatrial devices can selectively, fluidly connect a left atrium and a right atrium of the patient to facilitate blood flow therebetween. The interatrial devices can be adjustable closure devices, which in a first state prevent blood flow and in a second state permit (e.g., promote) blood flow. In some embodiments, the closure devices are adjustable to selectively control blood flow between the left atrium and the right atrium. For example, an adjustable closure device may include a closure element that is moveable between at least two (e.g., a plurality of) positions, that controls fluid flow through the closure device. In some embodiments, the closure devices described herein can include an actuator mechanism operably coupled to the closure element to actively move the closure element between the plurality of positions to control blood flow between the left atrium and right atrium. The adjustment can include an adjustment to a geometry of the lumen, or a geometry of an orifice (e.g., end flow aperture) of the lumen. The geometry can include a cross-section, an outer surface, an inner surface, a perimeter and/or a diameter. In some embodiments an end flow aperture is integrally formed with the lumen. In some embodiments, an end flow aperture is a device that is adjacent (e.g., coupled) to the lumen. In some embodiments, the adjustment occurs automatically without the need for active involvement of either the patient or a healthcare provider. In some embodiments, a closure element comprises a flow control element. In some embodiments, the adjustment requires an action from a user (e.g., a physician). For example, a user can change a value on an application or electronic interface, or a user can place a magnetized or other adjustment-initiation module proximate to the shunt. Additional features and examples of shunts having flow control mechanisms are described in International Patent Application No. PCT/US2020/038549, the disclosure of which is incorporated by reference herein in its entirety.

Some embodiments of the present technology adjust the closure elements to change a state of the closure device. For example, the closure device can have a first (e.g., baseline) state in which it is closed and does not allow blood flow between the LA and RA, and a second (e.g., active) state in which it is open and allows blood to flow between the LA and RA. In some embodiments, the closure element can include (a) an iris, (b) a hinge, (c) a strut, (d) a spindle, (e) a bladder, (f) a balloon, (g) a plug, and/or (h) a valve. The closure device can transition from the first state to the second state considering exertion level and/or heart rate of the patient, which may change due to exercise, stress, or other factors. The adjustment to the closure device can be made considering (e.g., based on, or in response to) sensed physiological parameters, including, for example, sensed LA pressure and/or RA pressure. In one example, when the LA pressure increases, the closure device can automatically open to allow blood flow to the RA. In some embodiments, the closure device can be configured to adjust based on, or in response to, an input parameter from a monitoring device. A monitoring device can be a pressure sensor. A monitoring device can be another device, such as a pulmonary arterial pressure sensor, insertable cardiac monitor, pacemaker, defibrillator, cardioverter, wearable, external electrocardiogram (ECG) or photoplethysmography (PPG), and the like. In some embodiments, the closure device is configured to remain closed until a predetermined threshold is reached, and once the threshold is reached, the closure device changes to an open state whereby it remains open. For example, the closure device may be configured to remain closed below a predetermined LA pressure threshold (e.g., 25 mmHg or 30 mmHg) and open above the predetermined LA pressure threshold. In some embodiments, the closure device is adjusted at a physician's discretion. In some embodiments, adjustments to the closure device are performed in a clinical setting.

Accordingly, as will be discussed in detail below, some embodiments of the present technology describe interatrial devices that are “smart” or otherwise dynamic in nature. In this context, the term “smart” may reference multiple variation approaches which are described in detail below and further understood from the description herein. For instance, a smart closure device may include sensors and/or information gathering capability in order to add diagnostic capability to the closure device, or to further enhance the therapeutic capabilities of the closure device. In other implementations, a smart closure may contain one or more lumens that are adaptable to selectively open or close in response to a patient's physiological condition or in response to a physician or healthcare provider action.

In embodiments including an active closure device, the perimeter (e.g., diameter) of a lumen may be changed by actively opening or closing portion(s) thereof, for example by adjusting a closure element that is coupled to the closure device. An end flow aperture (e.g., orifice) adjacent to or forming a portion of a lumen may be in fluid communication with the left atrium or the right atrium. Adjustments may be made in response to the operation of a motor or actuator, in response to a stimulus with mechanical, magnetic, or RF energy, or in response to another active stimulus. The shunt may include a battery or another integrated power source, or may externally powered via applied RF, Bluetooth, ultrasound, or other form of energy.

Some embodiments also include features and/or methods of use to prevent endothelialization, tissue overgrowth, and/or other phenomena that may lead to stenosis or occlusion of a lumen(s) of the closure device created via the implant or via the procedure. The features and/or methods of use described may be useful both to ensure the long-term patency of any fluid communication channels as well as to preserve the range of motion that is achievable by any of the components that are adapted to move, for example spindles that are adapted to flex, extend, and/or contract, or balloons that may inflate and deflate.

In one example of operation, closure devices configured in accordance with the present technology may have one or more adjustable or dynamic closure components move through a full range of motion at set intervals, for example, once every 6 hours, once every day, once every week, or another suitable period of time. For example, in embodiments that utilize one or more balloons to alter the diameter or effective diameter of a fluid communication channel, at set intervals the balloon may be inflated to its maximum capacity, then deflated to its minimum capacity, and then return to the desired volume. In a second example, embodiments that utilize valves or flaps may be forced open via electrical or mechanical means at set intervals. Some embodiments will be comprised or partially comprised of synthetic materials such as expanded Polytetrafluoroethylene (ePTFE) that limit or retard the onset of growth on implanted structures.

While certain embodiments of closure devices are described herein as fluidly connecting the LA and RA, one skilled in the art will appreciate that the devices can be configured to fluidly connect the LA and coronary sinus (CS) and include much of the same configurations and functionality. For example, in such embodiments, the sensors, components, and adjustable features of the implant or created-lumen may be similar to those described for interatrial embodiments.

### Control and Adjustment of Closure Devices

In some embodiments, the closure device is operably coupled to a controller. Suitable controllers include, for example, mobile device applications, computers, etc. The closure device may be coupled to the controller via Wi-Fi, Bluetooth (e.g., BLE 5.0), magnetic resonance, ultrasound, radiofrequency, or other wireless means. The closure device may be coupled to the controller via a wired connection. The controller provides a user interface such that a user (e.g., the patient, a physician, etc.) can selectively control the device via the controller. For example, a physician can input a desired flow rate and the controller can communicate with the closure device such that the actuator mechanism of the closure device manipulates the closure element to achieve flow.

The closure device may be coupled to the controller via a combination of wired and wireless connections. Distributing the communication across multiple devices and/or modalities may provide improved flexibility and power savings. It is understood that wireless communication with “deep” implants well below the skin-including the above closure device embodiment positioned in the heart-present greater challenges with wireless data and power transmission. The system in accordance with certain embodiments may utilize wireless technology to connect external components with a hub and wired technology to connect the hub to the closure device electronics. For example, the closure device may communicate to a subcutaneous device via a wired connection, and the subcutaneous device communicates to an external device via a wireless connection, or vice versa.

In some embodiments, the closure device is controlled using an adjustment module or tool. In some embodiments, the adjustment module or tool is actively powered. For example, the closure device can be configured such that the closure element can be adjusted via one or more energy modalities. A physician can then use the energy modality to manipulate the position of the closure element, thereby manipulating the flow between the LA and the RA. Suitable energy modalities can include, for example, magnetic, radiofrequency, ultrasonic, and the like.

In some embodiments, the energy source can be positioned external to the patient such that the energy is applied non-invasively. In other embodiments, however, the energy source can be positioned within the body (e.g., via a catheter) before targeting the device with the energy. In some embodiments, the energy is applied for a relatively short period of time (e.g., until the closure element is in the desired position), reducing the risk that tissue surrounding the device will overheat.

In some embodiments, the adjustment module is not actively powered and simply adjusts the closure element via purely mechanical means. In such embodiments, the adjustment module can be delivered to the device via a catheter.

In embodiments powered via magnetic energy, the closure device may include one or more magnetic coils. For example, a first magnetic coil can be positioned on a first side of a closure element and a second magnetic coil can be positioned on a second side of the closure element. The first magnetic coil and the second magnetic coil may be configured to be resonated, and therefore actuated, at different frequencies. Thus, the closure element can be adjusted by selectively targeting either the first magnetic coil or the second magnetic coil with their respective resonance frequencies. Suitable magnetic coils can be sized to fit in or on the closure device and to provide a target range of motion. For example, the coils can be about 2 cm or less.

In embodiments powered via radiofrequency (RF) energy, the closure device may have an antenna (e.g., a nitinol receiving antenna) configured to receive RF energy. The received energy can be used to adjust the closure element. In some embodiments, for example, the RF energy is delivered at a low frequency to reduce signal attenuation and/or to reduce tissue heating. Low frequency signals include signals having frequencies between about 20 kHz and 300 kHz. However, one skilled in the art will appreciate that other frequencies, such as those less than 20 kHz or greater than 300 kHz, may be used in certain embodiments of the present technology. In some embodiments, the received RF energy may comprise about 10-30 watts. Due to scattering attenuation, however, the device may receive less power than transmitted. Accordingly, the device can be configured to operate with less power than transmitted, such as one watt.

In embodiments powered via ultrasonic energy, the closure device may include various mechanical elements that move in response to exposure to ultrasound energy. For example, the closure element may be operably coupled to an ultrasonically actuatable element such that, when ultrasound energy is applied to the closure device, the closure element is adjusted.

In some embodiments, the actuator mechanism can be a motor. Suitable motors include electromagnetic motors, implanted battery and mechanical motors, MEMS motors, Maxon motors, piezoelectric based motors, and other motors capable of adjusting a closure element such as an iris may be used. In addition, other materials that can convert energy to linear motion can be used (e.g., nitinol). In some embodiments, a nitinol element is coupled to a pall or other mechanical element that is moved via actuation of the nitinol element.

As noted above, some embodiments of the closure devices include an electromagnetic motor. A variety of electromagnetic motors can be incorporated into the closure devices described herein to power the closure element. In some embodiments, for example, the closure device includes elements that actuate in response to electromagnetic energy or a change to a magnetic field. For example, in some embodiments, a “squirrel-cage” induction motor is incorporated into the device and operably coupled to the closure element. Applying a magnetic field across the closure device can cause a rotor of the squirrel-cage induction motor to spin, which in turn can adjust a position of the closure element.

As noted above, some embodiments of the closure devices include a battery coupled to a mechanical motor. The battery can be (a) incorporated into the closure device, (b) implanted yet spaced apart from the closure device, or (c) external to the patient. The battery can be operably coupled to the mechanical motor (e.g., a piezoelectric motor) such that the motor can be selectively actuated by the battery. In some embodiments, the battery can be controlled by incorporating metal-oxide-semiconductor field-effect transistors (“MOSFET”).

As noted above, some embodiments of the closure devices include MEMS motors. In some embodiments, for example, the MEMS motor may comprise a MEMS-based valve. The MEMS-based valve may sense flow at a microfluidic level and may function via thermal actuator principles to adjust a position of the valve to change flow therethrough. In some embodiments, the MEMS motor can be amplified with hydraulics. In some embodiments incorporating MEMS motors, the closure element may be biased towards a first position (e.g., a closed position) via a friction fit. The MEMS motor can be used to overcome the friction force to move the closure element towards a second position (e.g., an open position). As one skilled in the art will appreciate from the disclosure herein, a number of modifications to the motors described herein could incorporated into the closure devices without departing from the scope of the present technology.

### Select Embodiments of Adjustable Closure Devices

The closure devices described herein can include “active” devices. Active devices can incorporate a motor or other means to actively drive the closure element through a plurality of positions to alter (e.g., prevent or permit) blood flow between the LA and RA. Some embodiments of the present technology include both passive and active components.

In some embodiments, an implant (e.g., closure device) is configured to have no shunting—i.e., there is no lumen during normal modes of operation. The implant may function as a closure to a transseptal opening. The transseptal opening may be from a pre-existing condition (e.g., patent foramen ovule), or may have been introduced during an interventional procedure for a patient. In some embodiments, the implant may include or be operably coupled to one or more sensors for detecting one or more physiological properties (e.g., parameters) of the heart, such as LA blood pressure, RA blood pressure, flow velocity, heart rate, cardiac output, and/or myocardial strain. The sensors can be, for example, (1) embedded in the implant, (2) implanted along with yet spaced apart from the implant (e.g., in the LA, RA, CS, etc.), and/or (3) included on a wearable patch or device external to the body. In some embodiments, the wearable patch or device can also read sensor data. A sensor can be continuously recording or can be activated at select times. In one embodiment, for example, a sensor is battery powered and the battery is recharged via power harvesting. The sensors can transmit sensed physiological parameters to external display elements, external controllers, closure device control circuitry included on the device, and/or closure device control circuitry wirelessly coupled to the device.

In some embodiments, an implant that has no interatrial passageway in a first state is adaptable (e.g., adjusted) such that a passageway (e.g., lumen) is formed in a second state. The implant can be adjusted, for example via adjustment to a closure element or expansion with a balloon dilator, in order to facilitate future transseptal procedures. In some embodiments, the closure device includes a shunt passage which is selectively sealed (e.g. by an inflatable balloon). The seal can be removed (e.g. by deflating the balloon) thereby opening the passageway to facilitate delivery of therapeutic devices therethrough. For example, the shunt may be configured to open for the purpose of allowing introduction of a delivery tool and prosthetic valve from the RA to the LA. In still another embodiment, the implant may contain a sealed lumen that is initially blocked with one or more membranes that prevent fluid communication between heart chambers. If a future transseptal procedure is needed, a cutting or dilating catheter may be utilized to remove, puncture, or otherwise manipulate the membranes to establish a pathway into the LA from the RA. Following the procedure, either the lumen may be left open, or the lumen may be closed, for example using tools and techniques that are similar to those utilized during closure of patent foramen ovule (PFO).

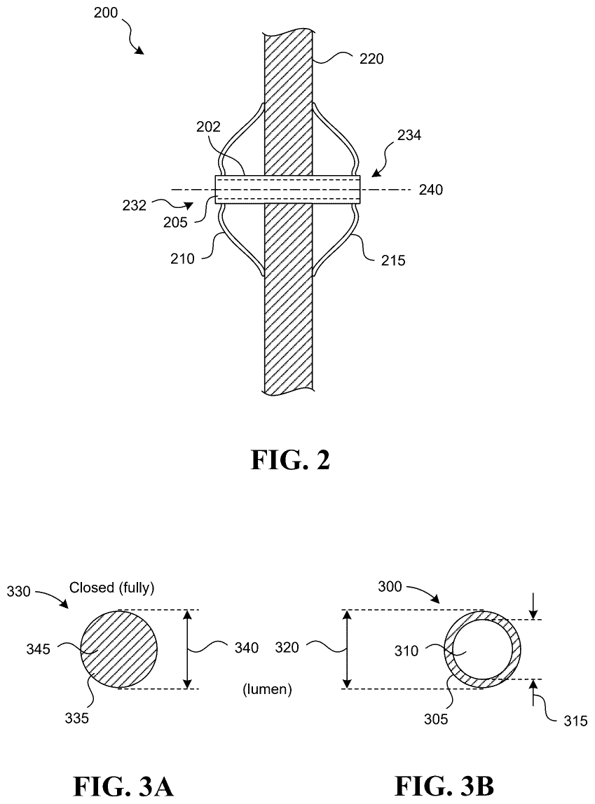

FIG. 2 illustrates an adjustable interatrial closure device 200 (“device 200”) implanted in native tissue of a patient and configured in accordance with embodiments of the present invention. As depicted in FIG. 2, the device 200 includes an outer tubular element 202 in contact with native tissue 220, a first anchoring element 210, a second anchoring element 215, and an adjustable inner surface (e.g., lumen) 205. The outer tubular element can be encased in an outer membrane that is suitable to engage native heart tissue. For example, the outer membrane can be a biocompatible and/or anti-thrombogenic material, such as ePTFE. In some embodiments, the outer membrane is an elastomeric material that is at least partially stretchable and/or flexible. In some embodiments the outer tubular element includes a plurality of arms defining an outer scaffolding for the device. An anchoring element can be coupled to the outer tubular element. An anchoring element can include a plurality of anchors. In some embodiments, the anchoring elements form part of the outer scaffolding for the closure device. The anchors can be configured to engage native heart tissue when the closure device is implanted in a heart, for securing the closure device in place.

The adjustable inner surface includes a proximal end portion 232 and a distal end portion 234. In some embodiments, the proximal end portion and the distal end portion are positionable within the right atrium and left atrium, respectively, when implanted in a human heart. A lumen can be sized such that, when implanted, the lumen fluidly connects the LA and the RA. The adjustable inner surface 205 extends the longitudinal length of the device 200 along a longitudinal axis 240. Accordingly, the adjustable inner surface fluidly connects the left atrium and the right atrium of the heart when implanted and in an open configuration (e.g., a second state).

In some embodiments, the adjustable lumen (e.g., inner surface) is selectively adjusted while the outer surface of the closure device retains a substantially constant outer geometry (e.g., perimeter). For example, the inner surface may be adjusted from a first state in which its inner perimeter is substantially zero (e.g., there is no lumen), to a second state in which the inner perimeter is nonzero (e.g., a lumen is opened), while an outer perimeter of the outer surface remains substantially unchanged. In some embodiments, an adjustment to an inner perimeter is made about a (e.g., central) axis of the closure body (e.g., FIG. 2, 240). In some embodiments, the adjustment is symmetric about the axis—e.g., the inner perimeter can be adjusted to increase or decrease concentrically with respect to the central axis. In some embodiments, the adjustment is asymmetric about the axis—e.g., the inner perimeter is adjusted non-centrically with respect to a central axis. For example, a closure element may adjust the inner surface to open or close from substantially one side (e.g., a superior or an inferior side) of a closure device body.

FIGS. 3A-3B illustrate in cross-section a closure device in a first state and a second state, according to embodiments of the present disclosure. In the example FIG. 3A, a closure device 330 in a first (e.g., closed) state comprises an outer surface 335 having an outer perimeter (e.g., diameter) 340, and a closed inner region 345 (e.g., that lacks a lumen). In the example of FIG. 3B, a closure device 300 in a second (e.g., open) state comprises an outer surface 305 having an outer perimeter 310, and an inner surface 310 having an inner perimeter (e.g., diameter) 315. The closure devices 300 and 330 may be the same closure device, and the outer perimeters 310 and 340 may be the same outer perimeter.

Using Sensors with Adjustable Closure Devices

In some embodiments the closure device is operably coupled to one or more sensors. The one or more sensors can be configured to detect a physiological parameter of the patient (e.g., LA pressure, RA pressure, heart rate, etc.) and transmit the detected physiological parameter to processing circuitry. A housing that house the sensor(s) can further include a transmitter. In some embodiments, a transmitter is disposed apart from the sensor(s). The processing circuitry can be included on the closure device, or can be external to the patient (e.g., an external display element or a controller). The processing circuitry receives the physiological parameter and determines, based at least in part on the parameter, if flow between the LA and RA needs to be adjusted. For example, the processing circuitry can determine if flow should be established (e.g., permitted) between the LA and the RA. In the case where flow adjustment is determined to be necessary, the processing circuitry can (e.g., automatically) perform an action. The action may be a notification (e.g., to a physician), or a command to one or more components (e.g., of the closure device). For example, a command to the closure device can comprise a direction to an actuator mechanism to change a position of the closure element (e.g., opening or closing the lumen) to achieve flow between the LA and the RA. In some embodiments, the adjustment may be made based upon a single parameter (e.g. the LA pressure alone), without regard for other parameters that may also be measured.

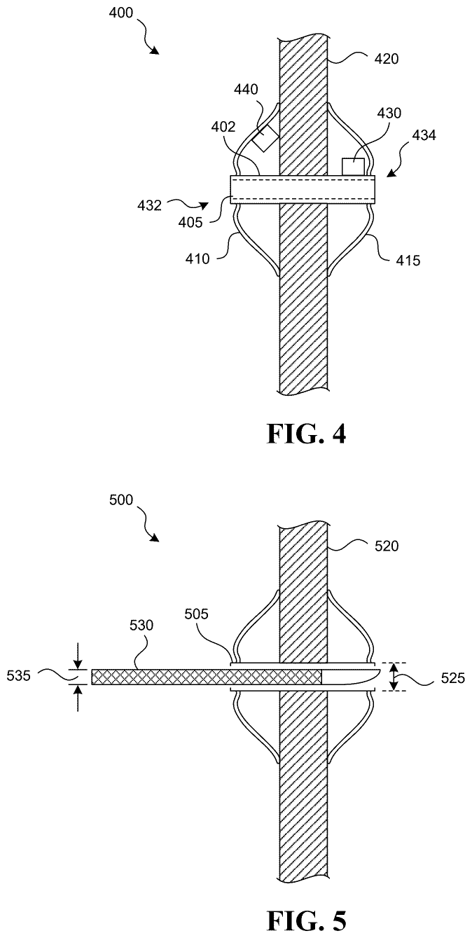

FIG. 4 illustrates an example adjustable interatrial closure device that includes sensors, implanted in native tissue of a patient and configured in accordance with embodiments of the present invention. As depicted in FIG. 4, the device 400 includes an outer tubular element 402 in contact with native tissue 420, a first anchoring element 410, a second anchoring element 415, and an adjustable inner surface (e.g., lumen) 405. The adjustable inner surface includes a proximal end (e.g., right atrium) portion 432 and a distal end (e.g., left atrium) portion 434. In some embodiments, the proximal end portion and the distal end portion are positionable within the right atrium and left atrium, respectively, when implanted in a human heart. A lumen can be sized such that, when implanted and in a second state, the lumen fluidly connects the LA and the RA. In the example of FIG. 4 the closure device includes two sensors-a sensor 430 is coupled with an anchoring element attached to native tissue, and a sensor 440 that is coupled to an outer surface of the closure device. A closure device can include one or more sensors. The sensor(s) may be coupled (a) directly to, or (b) at a remove from (e.g., not in direct contact with), the closure device. The sensors can be located within the LA, the RA, and/or a transseptal space—e.g., within the lumen, or positioned at a superior or inferior aspect of the closure device.

The sensor(s) can be encased in an outer membrane that is suitable to engage native heart tissue. For example, the outer membrane can be a biocompatible and/or anti-thrombogenic material, such as ePTFE. In some embodiments, the outer membrane is an elastomeric material that is at least partially stretchable and/or flexible. In some embodiments the sensor(s) are coupled with an outer scaffolding of the closure device.

In some embodiments, the closure device can automatically adjust a closure element in response to the sensed data. For example, if LA pressure increases above a predetermined threshold, the closure device can be (e.g., automatically) opened to provide a lumen extending between the LA and RA to permit blood flow (e.g., moving from the configuration illustrated in FIG. 3A to the configuration illustrated in FIG. 3B). Likewise, if LA pressure decreases below a predetermined threshold, the closure device can automatically decrease the inner diameter of the lumen to decrease blood flow (e.g., moving from the configuration illustrated in FIG. 3B to the configuration illustrated in FIG. 3A). In some embodiments, if the patient's heart rate increases above a predetermined threshold, the closure device can automatically increase an inner diameter of a lumen extending between the LA and RA to increase blood flow. Likewise, if the patient's heart rate decreases below a predetermined threshold, the closure device can automatically decrease the inner diameter of the lumen to decrease blood flow. In this manner, the closure device can increase exercise capacity.

A closure device may be implanted through a number of methods. In one embodiment, a method of implantation includes the following steps: (1) accessing the right atrium via a catheterization of a femoral, internal jugular vein, or another suitable vein; (2) optionally (e.g., when a transseptal opening is not already present), via a guidewire or other tools well-known in the field of interventional cardiology, navigating a suitable cutting catheter and/or transseptal puncture tools to the right atrium to form a transseptal puncture using established techniques; (3) with or without the exchange of a cutting catheter for a closure device delivery catheter, deploying the closure device, for example by traversing a catheter through the transseptal puncture into the left atrium, deploying a first portion of the closure device, retracting the catheter back into the right atrium, and deploying a second portion of the closure device; and (4) optionally, adjusting the closure device to be in a first state or a second state.

In some embodiments, a closure device is implanted for closing a pre-existing opening in a heart. For example, the pre-existing opening may be a congenital condition (e.g., atrial septal defect or ventricular septal defect), or may develop after birth (e.g., patent foramen ovale). The pre-existing opening may have been formed during a transseptal interventional procedure. In some embodiments, a closure device can be implanted using a surgical approach. In some embodiments, a closure device can be implanted using a transcatheter approach. In some embodiments, access for a transcatheter approach may be via femoral vein, internal jugular vein, or another suitable vein. In one embodiment, a method of implantation of a closure device in an atrial septal wall includes the following steps: (1) accessing the right atrium via a catheterization with a delivery catheter; and (2) deploying the closure device into the atrial septal wall. For example, the closure device can be deployed by traversing the delivery catheter through the transseptal opening into the left atrium, deploying a first portion of the closure device in the left atrium, retracting the delivery catheter back into the right atrium, and deploying a second portion of the closure device into the right atrium.

In some embodiments, a method of implantation of a closure device in a ventricular septal wall includes the following steps: (1) accessing the right ventricle via a catheterization with a delivery catheter; and (2) deploying the closure device into the ventricular septal wall. For example, the closure device can be deployed by traversing the delivery catheter through the transseptal opening into the left ventricle, deploying a first portion of the closure device in the left ventricle, retracting the delivery catheter back into the right ventricle, and deploying a second portion of the closure device into the right ventricle.

In some embodiments, a method of implantation of a closure device includes the following steps: (1) accessing the right atrium via a catheterization of a femoral, internal jugular vein, or another suitable vein; (2) when a transseptal opening is not already present, via a guidewire or other tools well-known in the field of interventional cardiology, navigating a suitable cutting catheter and/or transseptal puncture tool to the right atrium to form a transseptal puncture using established techniques; (3) with or without the exchange of a cutting catheter for a closure device delivery catheter, deploying the closure device. Deployment of the closure device can include traversing a catheter through the transseptal puncture into the left atrium, deploying a first portion of the closure device, retracting the catheter back into the right atrium, and deploying a second portion of the closure device.

In some embodiments, a closure device is delivered such that, following implantation, it remains in a first state (e.g., closed to fluid flow). In some embodiments, a closure device is delivered such that, following implantation, it remains in a second state (e.g., open to fluid flow). The closure device may be adjusted (e.g., by a physician) during implantation to place the closure device into the first or the second state.

According to some embodiments of the present disclosure, a closure device is able to accommodate surgical tools for transseptal access. In some embodiments, a closure device is placed into a third state (e.g., a maintenance state), in which the opening of the closure device is larger than that of the second state, for accommodating surgical tools for transseptal access. In some embodiments, the opening of the closure device in the second state is sufficient for accommodating surgical tools for transseptal access. A (e.g., smallest cross-sectional) size of an opening (e.g., lumen) of the closure device in the maintenance state can any value between approximately 6 mm to approximately 12 mm. The closure device can be adjusted into the maintenance state (or second state) (1) prior to introduction of the surgical tool, or (2) by (e.g., opening forces applied by) the surgical tool. FIG. 5 depicts an example of a closure device 500 having an outer surface 505 engaging native tissue 520, and accommodating a surgical (e.g., catheter) tool 530 for passing therethrough. In the example of FIG. 5, the closure device includes an inner surface (e.g., lumen) having an inner perimeter (e.g., diameter) 525 that is larger than an outer perimeter (e.g., diameter) 535 of the surgical tool.

In some embodiments, the closure device includes an adjustable inner lumen defined by a plurality of struts extending along the axial length of the lumen. The plurality of struts can be generally parallel to a center axis of the lumen. The struts can comprise a shape-memory material (e.g., nitinol). The lumen can be further defined by an inner membrane. In some embodiments, the inner membrane forms a sheath around the struts (e.g., the struts can be embedded within the inner membrane). In other embodiments, the struts can be positioned adjacent to but not encased within the inner membrane. For example, the struts can be internal to the inner membrane (e.g., within the lumen) or external to the inner membrane (e.g., outside the lumen). When the struts are not encased within the inner membrane, the struts can be otherwise connected to the inner membrane, although in other embodiments the struts are not connected to the inner membrane. Regardless of the relative positioning of the struts and the inner membrane, the inner membrane can form a single and/or continuous membrane with the outer membrane of the outer tubular element (in such embodiments, the outer membrane and the inner membrane can be collectively referred to as a single or unitary membrane). The volume of space between the outer membrane and the inner membrane can form a generally toroidal shaped chamber, as described in greater detail below. The inner membrane can comprise the same material as the outer membrane of the outer tubular element. For example, the inner membrane can be a biocompatible and/or anti-thrombogenic material such as ePTFE and/or an elastomeric material that is at least partially stretchable and/or flexible. For example, in one embodiment, the inner membrane is ePTFE and forms a sheath around the struts. In some embodiments, the inner membrane and the outer membrane can comprise different materials. In some embodiments, the device has two, three, four, five, six, seven, eight, nine, ten, eleven, and/or twelve struts.

The struts can be malleable and/or contain one or more hinges. A malleable strut enables the struts to dynamically change shape (e.g., expand, fold, or otherwise bend). A change in strut shape can promote a change in a perimeter (e.g., diameter) of the inner lumen. A volume between the outer membrane of the outer tubular element and the inner membrane of the adjustable inner lumen can define a generally toroidal shaped chamber. The chamber can be fluidly isolated from the interior of the lumen via the inner membrane. A chamber can also be fluidly isolated from the environment surrounding the device via the outer membrane. Accordingly, in some embodiments, the device is configured to prevent blood from flowing into the chamber. In some embodiments, the chamber can contain a compressible and/or displaceable liquid, gas, and/or gel. Accordingly, as the diameter of the lumen is adjusted, the liquid or gas can either be compressed, expanded, and/or displaced.

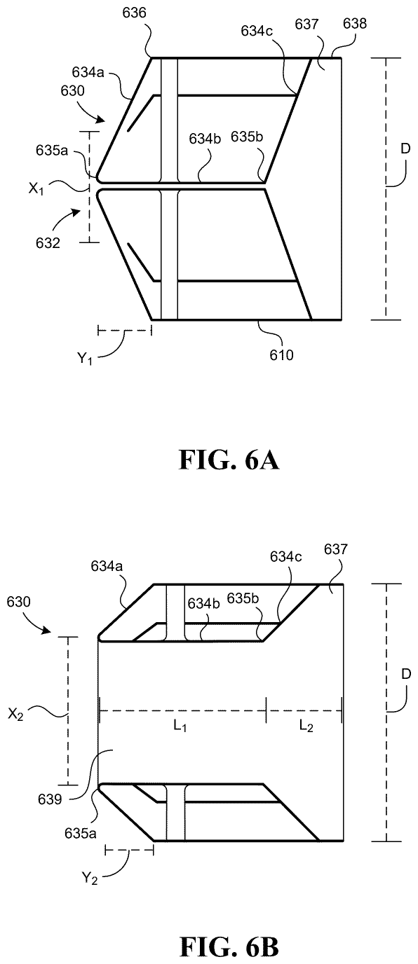

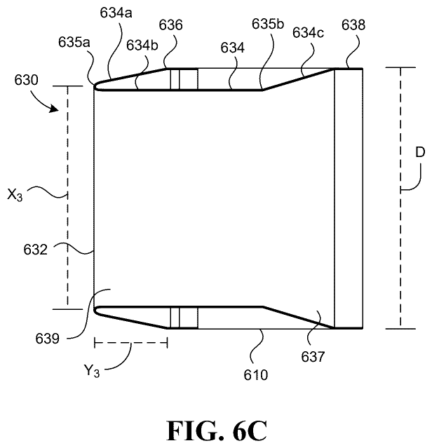

FIGS. 6A-6C schematically illustrate a closure device (e.g., in cross section) having an adjustable inner lumen 630 according to some embodiments of the present disclosure. Referring to FIG. 6A, struts 634 include a proximal strut (e.g., right atrium) segment 634a, a medial strut segment 634b, and a distal strut (e.g., left atrium) segment 634c. As illustrated, the medial strut segment 634b defines the lumen 630. The proximal strut segment 634a can connect the struts 634 to an outer tubular element 610 at a proximal connection 636 on the right atrium side of the closure device. The distal strut segment 634c can connect the struts 634 to the outer tubular element at a distal connection 638 on the left atrium side of the closure device. The transition between the proximal strut segment 634a and the medial strut segment 634b can include a hinge or other bendable aspect 635a (referred to hereinafter as “hinge 635a”). Likewise, the transition between the medial strut segment 634b and the distal strut segment 634c can include a hinge or bendable aspect 635b (referred to hereinafter as “hinge 635b”). The hinges enable strut segments to bend and/or fold relative to one another, thereby dynamically adjusting the perimeter (e.g., diameter) of the inner lumen 630, for example from a first (e.g., closed) state to a second (e.g., open) state.

Referring to FIG. 6A, a closure device is shown in a first configuration in which a portion of an inner lumen 630 defined by medial strut segments 634b is closed (e.g., in a first state), having a first inner perimeter X₁ that is substantially zero. An outer tubular element 610 has a perimeter D, and a proximal connection 636 and a hinge 635a are separated by a distance Y₁. To transition from the first state to the second state—e.g., to open the closure device—an inner perimeter of the lumen may be increased. In some embodiments, an increase in the inner perimeter of the lumen is generated when a proximal end portion (e.g., 632) of the inner lumen moves proximally, causing the struts (e.g., 634a-c) to bend at hinges (e.g., 635a-b). More specifically, in the illustrated embodiment, (a) the angle defined by the proximal strut segment 634a and the medial strut segment 634b at hinge 635a is decreased, and (b) the angle defined by the medial strut segment 634b and the distal strut segment 634c at hinge 635b is increased.

FIG. 6B illustrates a second configuration of the closure device in which the inner lumen 630 has a second inner perimeter X₂ that is greater than the first inner perimeter X₁. In the example of FIG. 6B, the proximal connection 636 and the hinge 635a are separated by a distance Y₂ that is greater than the distance Y₁. The perimeter D of the outer tubular element does not substantially change with an adjustment from the first configuration to the second configuration. FIG. 6C illustrates a third configuration of the closure device in which the inner lumen 630 is open (e.g., in the second state). The inner lumen may have a third inner perimeter X₃ that is greater than the first and second perimeters. In the example of FIG. 6C, the proximal connection 636 and the hinge 635a are separated by a distance Y₃ that is greater than the distance Y₂. The perimeter D of the outer tubular element does not change with an adjustment to the inner lumen, e.g., from the first configuration to the third configuration. As discussed above, the struts can comprise a shape memory material. Accordingly, once the struts have been transitioned to a desired position, the struts can retain their configuration and the lumen retains a constant perimeter until an active input is received (e.g. via an actuation mechanism, as discussed herein).

Although FIGS. 6A-6C only illustrate three lumen perimeters X₁-X₃, one skilled in the art will appreciate that the struts 634 can be actuated through a plurality of configurations (not shown), resulting in a plurality of lumen perimeters (not shown). For example, the lumen can take any perimeter between a fully closed configuration (e.g., X₁) and a fully open configuration (e.g., X₃). In various embodiments, closure device is configured to adjust from a first configuration to a second configuration. In some embodiments, the inner lumen has a substantially constant perimeter (e.g., along a longitudinal axis). The lumen may have a substantially constant perimeter along its entire length. In some embodiments, the lumen may have a substantially constant perimeter along (e.g., only) a major portion of its length. For example, the lumen perimeter may be substantially constant along the portion which extends through a septal wall. In another example, the lumen has a substantially constant perimeter along its entire length (e.g., FIG. 6B, L₁) except for features on one or both ends, such as a flare, funnel, taper, or the like (e.g., FIG. 6B, L₂). For example, a closure device shows the lumen 630 having a funnel shaped inflow portion (e.g., FIGS. 6A-6C, 637) configured for fluid communication with a left atrium of a heart (not shown) and a cylindrical shaped outflow portion (e.g., FIGS. 6B and 6C, 639) configured for fluid communication with the right atrium of a heart (not shown). In the illustrated embodiment of FIG. 6B, the cylindrical shaped outflow portion 639 extends along a major portion L₁ of the length of the lumen 630.

In some embodiments, the struts can be selectively actuated to increase the perimeter of the lumen. Referring to the example FIG. 6A, the perimeter of the inner lumen 630 can be increased by proximal movement of the proximal end portion 632 (e.g., further into the right atrium), such that (a) the angle defined by the proximal strut segment 634a and the medial strut segment 634b at hinge 635a is decreased, and (b) the angle defined by the medial strut segment 634b and the distal strut segment 634c at hinge 635b is increased. Accordingly, closure device enables the perimeter of the inner lumen to by selectively adjusted to control (e.g., promote) the flow of blood through the lumen. The specific perimeter for the inner lumen can be selected considering (e.g., based on) the patient's needs.

The closure device can be adjusted using an actuation mechanism (not shown). In some embodiments, the actuation mechanism is included on the closure device and can actively adjust the inner lumen perimeter. In some embodiments, for example, the actuation mechanism, when actuated, moves the proximal end portion 632 distally (e.g., and to open the lumen), causing the struts to bend as described above with respect to FIGS. 6A-6C. The actuation mechanism can also be configured to directly bend the struts 634 to alter the perimeter of the lumen 630.

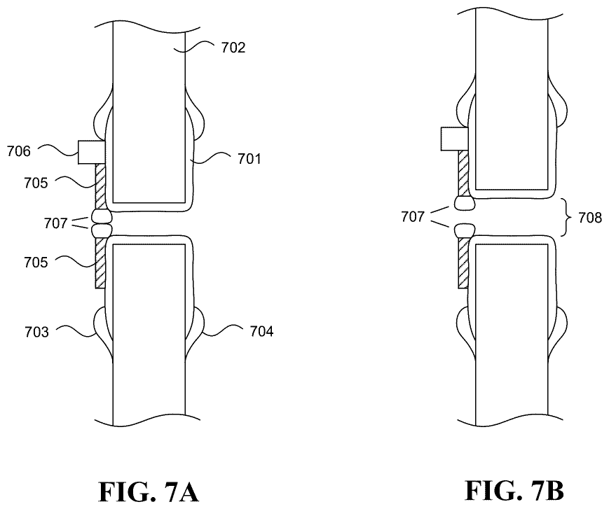

FIGS. 7A and 7B illustrate another embodiment of an adjustable closure device of the present technology. In this embodiment, the effective perimeter of an inner surface (e.g., lumen) in the closure device may be adjusted outside of a lumen. For example, one or more closure elements may be disposed in only one chamber of the heart (e.g., sitting at a proximal end or a distal end of a closure device). For example, a balloon or a bladder may be positioned such that expansion and contraction restrict (e.g., prevent) and promote (e.g., permit), respectively, fluid flow through the closure device. In FIG. 7A, a transverse view of a closure device in septum 702 is shown in a first (e.g., initial) state. The closure device can be implanted to be positioned on both sides of the septum, with a central opening of the implant providing a pathway that enables interchamber fluid communication (e.g., in a second state). The example closure device 701 includes RA-side anchors 703 and LA-side anchors 704 which hold the implant in place. On the RA side, the implant may contain additional components, such as one of more filling pumps (e.g., 706), one or more filling conduit lines (e.g., 705), and one or more narrowing balloons (e.g., 707). In the first state shown in FIG. 7A, the narrowing balloons 707 are in a (e.g., expanded) geometric configuration that substantially closes an end of the closure device, preventing fluid flow therethrough.

FIG. 7B illustrates another configuration of the embodiment of FIG. 7A. At the discretion of a healthcare provider or in response to a change in a physiological parameter (e.g., an increased pressure differential detected by one or more pressure sensors included in the implant (not shown)), the closure device may be adjusted from the first state to the second state. In the example of FIG. 7B, narrowing balloons 707 have decreased in geometric size, thereby opening a lumen 708 to allow fluid flow therethrough. In a mode of operation, a filling pump forces a filling substance (i.e. a gel, foam, gas, or fluid) through conduits and into compliant or semi-compliant balloons, which caused the balloons to grow in size. The filling pump may also be capable of removing a filling substance from the balloons in order to reduce them in size. In some implementations the pump itself has some storage capacity to hold filling substances when the balloon in configured in smaller geometries. In alternative implementations, a secondary storage component such as a bladder (not shown) or secondary balloon may be used to hold filling substance when it is not contained within the narrowing balloons. In some embodiments, a battery or another source of active power may be included on or proximate to the closure device in order to facilitate operation of the filling pump and/or other implant components, such as any sensors that are included on the implant.

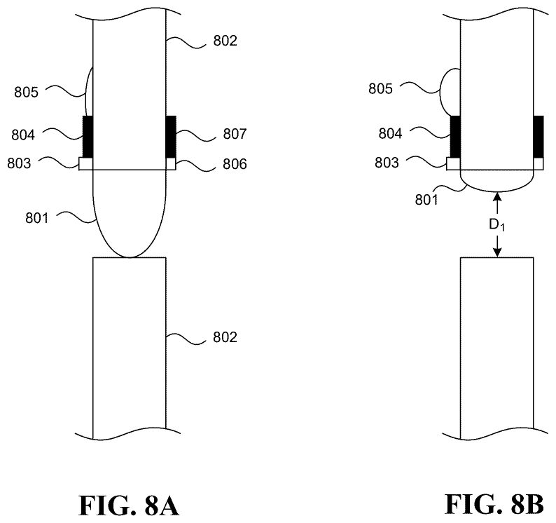

FIGS. 8A and 8B illustrate a closure device configured in accordance with select embodiments of the present technology. The closure device is placed to have an adjustment element biased toward one portion of a transseptal opening (for example, at the superior or inferior aspect of the opening). The biased element can provide an asymmetric adjustment to the inner perimeter of closure device, e.g., an adjustment that is not concentric about a central axis of the transseptal opening and/or of the closure device. The adjustment element is operable to transition the closure device between the first state and the second state, e.g., by expansion and contraction. The closure device contains an expansion element (e.g., balloon), preferably of a compliant material comprised of latex, silicone, nylon, or another similar material. In the example FIG. 8A, a closure device in a first state includes a balloon 801 that completely occludes an opening in a septum 802. The exemplary closure device of FIG. 8A also contains a pumping mechanism 803, a proximal anchor 804, a secondary balloon 805, one or more pressure sensors 806, and a distal anchor 807. In embodiments, pressure sensors may be located in either chamber of the heart, or in the septal opening. In embodiments, the implant may contain other hardware features, for example a battery or other power source, battery recharging hardware and circuitry, a data communication means, on-board memory, and other appropriate components. In some embodiments, the transseptal opening is given structural support, for example by placing a metal stent or a bioabsorbable stent, or by treating the tissue at the perimeter of the opening with energy such as radiofrequency energy.

In FIG. 8B, an embodiment of the closure device is shown in the second state. Balloon 801 has been reduced (e.g., deflated), to produce a passage through the septal wall with secondary balloon 805 increased in size. In the second state, the passage has a (e.g., nonzero) perimeter (e.g., D1) that permits fluid flow between the LA and the RA.

In an example mode of operation, the implant generally is configured as shown in FIG. 8A, with the balloon 801 enlarged such that there is no passage through the transseptal opening. If pressure sensor 806 notes a left atrial pressure that exceeds a pre-determined value, remains elevated over pre-determined value for a pre-set period of time, or changes by a pre-determined amount over a pre-set period of time, on-board electronics instruct pumping mechanism 803 to initiate a transfer of balloon media (e.g., air, saline, or another gas, gel, foam, or fluid) from balloon 801 to secondary balloon 805, thus reducing the volume of balloon 801 and thereby opening the septal opening to have an effective perimeter (e.g., D1). This widening of the septal opening allows flow between heart chambers, and may effectively unload the left atrium. Based on readings from pressure sensor 806, other sensors, after a set period of time, or based on another criteria, pumping mechanism 803 may reverse the filling states of the balloons and return the implant to its original (e.g. closed) configuration. In variation embodiments, balloon 801 may operate in a continuum of inflation states and sizes to create a spectrum of possible opening perimeters.

Closure Device with Adjustable Inner Diameter to Allow for Heart Procedures

In further embodiments, a lumen of an implant or the lumen created with a procedure may be dilated or expanded to a size that is not typical for use with interatrial shunts in order to allow additional transseptal cardiac procedures to occur. In an embodiment, the lumen may be expanded up to 24 Fr, 30 Fr, 40 Fr, or larger. In embodiments, the lumen portion of an implant is adapted to be flexible such that it may be dilated with a cylindrical balloon in order to provide temporarily enlarged transseptal access. In such embodiments some or all materials comprising the lumen portion of an implant may be constructed of a material with shape-memory properties, such as nitinol. In variation embodiments, a shutter, iris, valve, or balloon configuration may have a normal diameter range of adjustable operation, which at the discretion of a physician may be opened to a wider diameter that is outside of this normal range in order to facilitate interventional tools traversing the lumen. Following the secondary procedure, the lumen diameter may be reconfigured to once again operate primarily within the normal range of diameters.

### EXAMPLES

Several aspects of the present technology are set forth in the following examples:
1. A method of reversibly closing a transseptal opening between a right atrium and a left atrium in a patient via a closure device, the method comprising:
implanting the closure device into the transseptal opening, wherein the closure device comprises a first end flow aperture in fluid communication with a left atrium, a second end flow aperture in fluid communication with a right atrium, and a lumen extending between the first end flow aperture and the second end flow aperture; and
adjusting the closure device to a first state or a second state, wherein adjusting to the first state comprises substantially preventing fluid flow through the lumen, and adjusting to the second state comprises promoting fluid flow through the lumen.
2. The method of example 1 wherein adjusting the closure device comprises altering a volume of an expandable chamber that is (i) adjacent to, or (ii) at least partially surrounding, the lumen.
3 The method of example 2 wherein the altering the volume of the expandable chamber comprises altering a length of the lumen along a longitudinal axis of the closure device.
4. The method of example 3 wherein the altering the volume of the expandable chamber comprises altering a position of the lumen along the longitudinal axis.
5. The method of any one of examples 2-4 wherein the first end flow aperture comprises a first opening perimeter, and the second end flow aperture comprises a second opening perimeter.
6. The method of example 5 wherein the expandable chamber is adjacent to the first end flow aperture or to the second end flow aperture, and wherein altering the expandable chamber comprises altering the first opening perimeter or the second opening perimeter.
7. The method of example 6 wherein the expandable chamber is adjacent to the second end flow aperture.
8. The method of example 2 wherein the adjusting comprises using a hydraulic force for altering the volume of the expandable chamber.
9. The method of example 8, further comprising imparting the hydraulic force through a liquid or a gel that is filling the expandable chamber.
10. The method of example 2 wherein the altering the volume of the expandable chamber comprises applying electromagnetic or ultrasonic energy to change a dimension of a spring or a coil that is coupled with at least a portion of the expandable chamber.
11. The method of example 10 wherein the spring or coil comprises a shape memory material.
12. The method of example 11 wherein the spring or coil comprises nitinol.
13. The method of example 1 wherein the adjusting comprises applying electromagnetic or ultrasonic energy to change a dimension of a spring or a coil that is coupled with at least a portion of the lumen.
14. The method of any one of examples 1-13 wherein the adjusting to the first state or the second state occurs while maintaining an outer geometry (e.g., perimeter) of the closure device.
15. The method of example 14, further comprising selecting the outer geometry of the closure device considering a size of the transseptal opening.
16. The method of example 15 wherein selecting the outer geometry occurs prior to implanting the closure device.
17 The method of example 15 wherein the outer geometry is modifiable, and wherein the method further comprises modifying the outer geometry during implanting of the closure device.
18. The method of any one of examples 1-17 wherein the adjusting is to the first state, prior to implanting the closure device.
19. The method of any one of examples 1-18 wherein the adjusting is to the second state, considering measured physiological data from a sensor that is operably coupled with the closure device.
20. A system for selectively closing a transseptal opening between a right atrium and a left atrium in a patient, the system comprising:
a closure device, comprising—
   an elongate body;
   a lumen extending along the elongate body; and
   a closure element coupled with the elongate body or the lumen; and
an actuator coupled with the closure element,
wherein the closure element is adjustable by the actuator to place the closure device into a first state or a second state,
wherein—
   in the first state, fluid flow through the lumen is prevented, and
   in the second state, fluid flow through the lumen is promoted.
21. The system of example 20 wherein the closure element comprises an expandable chamber, and wherein the adjustment to the closure element comprises an increase or a decrease to a volume of the expandable chamber.
22. The system of example 20 wherein the closure element is adjustable by the actuator in response to an input.
23. The system of example 22 wherein the actuator is electrically actuatable, and wherein the input comprises an electromagnetic signal, a radiofrequency signal, and/or an ultrasonic signal.
24 The system of any one of examples 20-23 wherein the closure element comprises a hinge, an iris, a shutter, a strut, a bladder, a spindle, or a valve.
25. The system of any one of examples 20-24 wherein the closure element comprises a shape memory material.
26 The system of example 25 wherein the closure element comprises nitinol.
27 The system of any one of examples 20-26, further comprising a pressure sensor for detecting one or more physiological properties of the patient.
28 The system of example 27 wherein adjustment to the first state or the second state is in consideration of a detected physiological property by the pressure sensor.
29. The system of example 27 wherein the pressure sensor is a first pressure sensor of at least two pressure sensors.
30. The system of example 29 wherein at least one of the at least two pressure sensors is disposed at or adjacent to the left atrium, the right atrium, the lumen, or a septal wall of the patient.
31. The system of example 28, further comprising a transmitter operably coupled with the pressure sensor, wherein the transmitter is adapted to transmit the detected physiological property to a processor.
32. The system of any one of examples 28-31 wherein the pressure sensor comprises a piezoelectric, MEMS, acoustic, or fluid column sensor.
33. The system of any one of examples 28-32 or wherein the closure device further comprises an anchor portion, wherein the pressure sensor is disposed adjacent the anchor portion.
34 A method of treating a patient, the method comprising:
advancing a closure device to a location near a transseptal opening between a right atrium and a left atrium of the patient;
positioning the closure device within the transseptal opening, the closure comprising an elongate body sized for filling the transseptal opening and preventing fluid communication therethrough;
positioning a sensor coupled with the closure device for measuring at least one physiological property in at least one of the right atrium and the left atrium, and storing the at least one measured physiological property in a memory; and
based, at least in part, on the measured physiological property, adjusting a lumen extending through the elongate body between a first state and a second state,
wherein, in the first state, the lumen substantially prevents fluid flow therethrough and, in the second state, the lumen passes fluid therethrough.
35. The method of example 34 wherein the sensor comprises a first sensor positioned in the left atrium for measuring a first physiological property in the left atrium, and the method further comprises positioning a second sensor for measuring a second physiological property in the right atrium.
36 The method of example 34, further comprising using a transmitter coupled with the closure device for transmitting the measured physiological property to a processor.
37 The method of example 36 wherein transmitting the measured physiological property comprises periodically transmitting.
38 The method of example 36 wherein transmitting begins upon activating the transmitter via a power source.
39 The method of example 38 wherein the activating is by a battery and/or a capacitor that is within or adjacent to a chamber of the heart.
40 The method of example 35 wherein the measuring comprises a first measuring within the right atrium and a second measuring within the left atrium.
41. The method of example 40 wherein the first measuring is performed by a first sensor, and the second measuring is performed by a second sensor.
42. The method of example 34 wherein the advancing comprises intravascularly advancing.
43. The method of example 34 wherein the advancing the closure device is following a transseptal interventional procedure.
44 The method of example 43 wherein the transseptal interventional procedure comprises a left atrial appendage ligation, pulmonary vein isolation, and/or mitral valve repair.
45 The method of example 43 wherein the advancing is without removing a previously-inserted catheter for performing the transseptal interventional procedure.
46. The method of example 34 wherein positioning comprises anchoring the closure device at or adjacent to the transseptal opening.
47. The method of example 46 wherein anchoring comprises deploying a self-expanding anchor.
48. A method of implanting a reversibly closable closure device into a heart of a patient, the method comprising:
implanting the closure device into the heart of the patient, wherein the closure device comprises a first end flow aperture in fluid communication with a left atrium, a second end flow aperture in fluid communication with a right atrium, and a lumen extending between the first end flow aperture and the second end flow aperture; and
adjusting the closure device to a first state or a second state, wherein adjusting to the first state comprises substantially preventing fluid flow through the lumen, and adjusting to the second state comprises promoting fluid flow through the lumen.
49 A closure device for reversibly closing a transseptal opening between a right atrium and a left atrium in a patient, the closure device comprising:
an elongate body sized for filling the transseptal opening; and
a lumen extending along the elongate body that, in a first state, is closed for preventing fluid flow therethrough, and, in a second state, is open for promoting fluid flow therethrough.
50. The closure device of example 49, further comprising a sensor operable to detect at least one physiological property in the right atrium and/or the left atrium.
51. The closure device of example 50, wherein the closure device further comprises a memory, and the sensor is communicatively coupled with the memory for transmission of a measured physiological property thereto.
52. The closure device of example 50 wherein the sensor is communicatively coupled with a receiver that is disposed external to the patient, and is operable for transmission of a measured physiological property thereto.
53 The closure device of example 50 wherein the sensor comprises a first sensor disposed in the left atrium, and wherein the closure device further comprises a second sensor disposed in the right atrium.
54 The closure device of example 49, further comprising an actuation mechanism for adjustment of the lumen between the first state and the second state.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the left atrium and right atrium, the left ventricle and the right ventricle, or the left atrium and the coronary sinus, it should be appreciated that similar embodiments could be utilized for shunts between other chambers of heart or for shunts in other regions of the body.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A closure device for reversibly closing a transseptal opening between a right atrium and a left atrium in a patient, the closure device comprising:
an elongate body sized for filling the transseptal opening; and
a lumen extending along the elongate body that, in a first state, is closed for preventing fluid flow therethrough, and, in a second state, is open for promoting fluid flow therethrough.

2. The closure device of claim 1, further comprising a sensor operable to detect at least one physiological property in the right atrium and/or the left atrium.

3. The closure device of claim 2, wherein the closure device further comprises a memory, and the sensor is communicatively coupled with the memory for transmission of a measured physiological property thereto.

4. The closure device of claim 2 wherein the sensor is communicatively coupled with a receiver that is disposed external to the patient, and is operable for transmission of a measured physiological property thereto.

5. The closure device of claim 2 wherein the sensor comprises a first sensor disposed in the left atrium, and wherein the closure device further comprises a second sensor disposed in the right atrium.

6. The closure device of claim 1, further comprising an actuation mechanism for adjustment of the lumen between the first state and the second state.

7. A system for selectively closing a transseptal opening between a right atrium and a left atrium in a patient, the system comprising:
a closure device, comprising—
an elongate body;
a lumen extending along the elongate body; and
a closure element coupled with the elongate body or the lumen; and
an actuator coupled with the closure element,
wherein the closure element is adjustable by the actuator to place the closure device into a first state or a second state,
wherein—
in the first state, fluid flow through the lumen is prevented, and
in the second state, fluid flow through the lumen is promoted.

8. The system of claim 7 wherein the closure element comprises an expandable chamber, and wherein the adjustment to the closure element comprises an increase or a decrease to a volume of the expandable chamber.

9. The system of claim 7 wherein the actuator is electrically actuatable in response to an input to adjust the closure element, and wherein the input comprises an electromagnetic signal, a radiofrequency signal, and/or an ultrasonic signal.

10. The system of claim 7 wherein the closure element comprises a hinge, an iris, a shutter, a strut, a bladder, a spindle, or a valve.

11. The system of claim 7 wherein the closure element comprises a shape memory material.

12. The system of claim 11 wherein the closure element comprises nitinol.

13. The system of claim 1, further comprising a pressure sensor for detecting one or more physiological properties of the patient.

14. The system of claim 13 wherein the pressure sensor is a first pressure sensor of at least two pressure sensors.

15. The system of claim 14 wherein at least one of the at least two pressure sensors is disposed at or adjacent to the left atrium, the right atrium, the lumen, or a septal wall of the patient.

16. The system of claim 13, further comprising a transmitter operably coupled with the pressure sensor, wherein the transmitter is adapted to transmit the detected physiological property to a processor.

17. The system of claim 13 wherein the pressure sensor comprises a piezoelectric, MEMS, acoustic, or fluid column sensor.

18. The system of claim 13 wherein the closure device further comprises an anchor portion, wherein the pressure sensor is disposed adjacent the anchor portion.
